SEQUENCE LISTING (10 sequences)

SEQ ID NO: 1 - DNA, 7053 bp, Artificial Sequence
  Synthetic cDNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 2 - DNA, 7053 bp, Artificial Sequence
  Synthetic cDNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 3 - DNA, 7053 bp, Artificial Sequence
  Synthetic cDNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 4 - DNA, 7053 bp, Artificial Sequence
  Synthetic cDNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccatcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 5 - DNA, 7053 bp, Artificial Sequence
  Synthetic cDNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacagtgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 6 - DNA, 7053 bp, Artificial Sequence
  Synthetic cDNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacggaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 7 - DNA, 9030 bp, Artificial Sequence
  Synthetic cDNA
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaacccccactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccatttggtcttaattctgaccaataaaaaaataagtcaggaggatgcaattgttgaaagctttgaaataaaataacaatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgaccccttcactatgactacacagaatctcctgatagtaaagggggctggaggcaaggataagttatagagcagttggaggaagcatccaaagattgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacataca

SEQ ID NO: 8 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPPLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - DNA, 9030 bp, Artificial Sequence
  Synthetic cDNA
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtcatgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccatcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgagggggcatatgctccagtacttcaagattttaggtcattaaatgattcaacaaataaaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactgagggtggccactgcagcacctgccactgccgtcacctctccctcctcagctccagggcagtgtccctccctggcttgccttctacctttgtgctaaatcctagcagacactgccttgaagcctcctgaattaactatcatcagtcctgcatttctttggtggggggccaggagggtgcatccaatttaacttaactcttacctattttctgcagctgctcccagattactccttccttccaatataactaggcaaaaagaagtgaggagaaacctgcatgaaagcattcttccctgaaaagttaggcctctcagagtcaccacttcctctgttgtagaaaaactatgtgatgaaactttgaaaaagatatttatgatgttaacatttcaggttaagcctcatacgtttaaaataaaactctcagttgtttattatcctgatcaagcatggaacaaagcatgtttcaggatcagatcaatacaatcttggagtcaaaaggcaaatcatttggacaatctgcaaaatggagagaatacaataactactacagtaaagtctgtttctgcttccttacacatagatataattatgttatttagtcattatgaggggcacattcttatctccaaaactagcattcttaaactgagaattatagatggggttcaagaatccctaagtcccctgaaattatataaggcattctgtataaatgcaaatgtgcatttttctgacgagtgtccatagatataaagccatttggtcttaattctgaccaataaaaaaataagtcaggaggatgcaattgttgaaagctttgaaataaaataacaatgtcttcttgaaatttgtgatggccaagaaagaaaatgatgatgacattaggcttctaaaggacatacatttaatatttctgtggaaatatgaggaaaatccatggttatctgagataggagatacaaactttgtaattctaataatgcactcagtttactctctccctctactaatttcctgctgaaaataacacaacaaaaatgtaacaggggaaattatataccgtgactgaaaactagagtcctacttacatagttgaaatatcaaggaggtcagaagaaaattggactggtgaaaacagaaaaaacactccagtctgccatatcaccacacaataggatcccccttcttgccctccacccccataagattgtgaagggtttactgctccttccatctgcctgaccccttcactatgactacacagaatctcctgatagtaaagggggctggaggcaaggataagttatagagcagttggaggaagcatccaaagattgcaacccagggcaaatggaaaacaggagatcctaatatgaaagaaaaatggatcccaatctgagaaaaggcaaaagaatggctacttttttctatgctggagtattttctaataatcctgcttgacccttatctgacctctttggaaactataacatagctgtcacagtatagtcacaatccacaaatgatgcaggtgcaaatggtttatagccctgtgaagttcttaaagtttagaggctaacttacagaaatgaataagttgttttgttttatagcccggtagaggagttaaccccaaaggtgatatggttttatttcctgttatgtttaacttgataatcttattttggcattcttttcccattgactatatacatctctatttctcaaatgttcatggaactagctcttttattttcctgctggtttcttcagtaatgagttaaataaaacattgacacataca

SEQ ID NO: 10 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVHPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNKTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2012/0297494 A1
(43) Pub. Date: Nov. 22, 2012

(54) COMPOSITIONS AND METHODS OF TREATMENT OF BLACK HEMOPHILIAC PATIENTS

(76) Inventor: Tommy Eugene Howard, Manhattan Beach, CA (US)

(21) Appl. No.: 13/502,292

(22) PCT Filed: Oct. 16, 2009
(86) PCT No.: PCT/US2009/061075

§ 371 Date: Apr. 16, 2012

(51) Int. Cl. A61K 38/37; A61P 7/04; A01K 67/00; C07K 14/755; C12Q 1/68

(52) U.S. Cl. 800/13; 514/14.1; 530/383; 435/6.11

(57) ABSTRACT

It has been determined that most mutations in factor VIII occur in multiple haplotypes, not primarily in one haplo-type. The frequencies of mild, moderate, and severe hemophilia did not differ significantly according to the background haplo-type. The odds of having inhibitor were significantly higher among patients in the H3+H4 haplotype groups as compared to H1+H2 haplotype groups. This association appears to be independent of the mutation. The results indicate that white hemophiliacs should be treated with Kogenate®. However, it would clearly be of benefit to assess the haplotype of black hemophiliacs prior to prescribing the recombinant FVIII to be used for treatment. It is not essential to determine the actual mutations responsible for the hemophilia prior to prescribing the recombinant FVIII. Also described are transgenic human FVIII animal models.

### STATEMENT REGARDING FEDERALLY SPONSORED RESEARCH OR DEVELOPMENT

The Government has certain rights in the invention since the invention was made with support from Grant Nos. HL-71130 and HL-72533 to Dr. Howard; Grant No. HL-07109 to Dr. Thompson and HL-70751 to Dr. Almasy by the National Institutes of Health.

### FIELD OF THE INVENTION

The invention is generally in the field of diagnostic and therapeutics for hemophiliacs.

### BACKGROUND OF THE INVENTION

Hemophilia is a congenital bleeding disorder. Patients with Hemophilia A have either absent, decreased or defective production of the blood clotting protein, Factor VIII (FVIII). Those with Hemophilia B have similar, problems with Factor IX (FIX). Hemophilia is characterized as “severe” when the activity of the affected clotting factor (FVIII or FIX) is less than 1% of normal. Severe Hemophilia is often associated with spontaneous bleeding (i.e. bleeding not caused by trauma or injury). Hemophilia is termed “mild” when the relevant clotting factor activity is 6-24% of normal. Hemophilia is referred to as “moderate” when clotting factor activity is between 1% and 5% of normal. Approximately 70% of Hemophilia patients have severe disease and can require treatment for bleeding several times per month.

Most patients that have Hemophilia A or B are treated by replacing their missing coagulation factor with FVIII or FIX that is either derived from plasma or developed using recombinant technology. Several recombinant F VIII preparations are available: Kogenate® (Bayer Healthcare), Recombinate® and Advate® Antihemophiliac F VIII (Baxter Healthcare), Refacto Antihemophiliac factor (3 domain deleted, Wyeth), and Helixate® (CSL Behring).

One of the most serious complications of the treatment of Hemophilia is the development of ‘inhibitors’ (see package insert for Recombinate® and Kogenate®). ‘Inhibitors’ are antibodies to FVIII or FIX that can develop in patients with Hemophilia following replacement therapy with the missing coagulation factor. The management of Hemophilia patients with inhibitors is difficult. Clinically, most inhibitors are detected when patients fail to respond to standard replacement therapy.

Inhibitors are usually first detected using a sensitive clotting-based assay, variably referred to as an inhibitor screen or a mixing study. The coagulation factor specificity of the suspected inhibitor is next commonly determined by performing a set of clotting-based factor activity assays where each is specific for one of the candidate coagulation proteins potentially being targeted. The presence and specificity of an inhibitor is most often confirmed by performing the more specific clotting-based test known as the Bethesda assay. The plasma level (i.e. titer) of an inhibitor is defined in terms of Bethesda units (BU). In general, a patient having a BU exceeding 10 is considered refractory to treatment with human FVIII.

The replacement clotting factors are typically obtained from human plasma or from recombinant (genetically engineered) preparations. Human plasma-derived clotting factors have the inherent risk of potentially transmitting certain viruses. Antibodies or ‘inhibitors’ can develop following treatment with either human plasma factor concentrates or recombinant clotting factor preparations. Alloantibodies react with the replacement fVIII product but not with the patient's endogenous fVIII. Occasionally patients develop autoantibodies in addition to alloantibodies in response to infused fVIII. When this occurs, a mild or moderate patient may become a severe patient. The development of inhibitors is very problematic as injected replacement therapy is frequently ‘neutralized’ or made ineffective by the inhibitor shortly after infusion. Treatment options available for treating Hemophilia patients that develop inhibitors include high dose FVIII or bypassing agents such as prothrombin complex concentrates (PCCs) or activated prothrombin complex concentrates (e.g., FEIBA and other APCCs) which enhance the hemostatic process without the need of FVIII or FIX.

The incidence of FVIII inhibitors in black patients is approximately twice that in whites. U.S. Ser. No. 11/720,945 filed Jun. 6, 2007 described the discovery that, in What may be a unique situation, FVIII appears to vary by haplotype based on ethnic origin—i.e., the majority of whites and Chinese have haplotype 1, blacks have haplotype 1, 2, or 3, and there are small numbers of individuals with haplotypes 4, 5, or 6. This discovery was based on analysis of factor VIII from normal individuals, not hemophiliacs. F VIII from 137 healthy people representing seven ethnic groups was sequenced. This identified four common nonsynonymous single nucleotide polymorphisms (nsSNPs). Naturally-occurring haplotypes of these nsSNPs encode six structurally distinct wildtype FVIII proteins. Five of these haplotypes, designated H1, H2, H3, H4 and H15, are expressed by African-Americans, whereas only two, H1 and H2, are expressed by Caucasians. Two haplotypes, H3 are H5, which together are expressed in approximately 23% of African-Americans, have the minor allele of M2238V in the C2 dominant epitope. The two commercially available recombinant FVIII cornpositions are haplotype 1 (Kogenate®) and haplotype 2 (Recombinatet). It was proposed that there would be a lower incidence of inhibitors if the patients were matched by haplotype with the replacement factor VIII. It was not known, however, whether this would actually occur with hemophiliacs, with all or some mutations, and whether or not the mutations might actually make haplotype irrelevant. It is well established in the literature that the type of mutation affects the incidence of inhibitors, although it is not known if this is independent of haplotype or not.

Therefore, it is an object of the invention to provide recombinant human factor VIII haplotypes for treatment of various mutations of factor VIII.

### SUMMARY OF THE INVENTION

It has been determined that most mutations in factor VIII occur in multiple haplotypes, not primarily in one haplotype. The frequencies of mild, moderate, and severe hemophilia did not differ significantly according to the background haplotype. The odds of having inhibitor were significantly higher among patients in the H3+H4 haplotype groups as compared to H1+H2 haplotype groups. This association appears to be independent of the mutation. The results indicate that white hemophiliacs should be treated with Kogenate®. However, it would clearly be of-benefit -to assess the haplotype of black hemophiliacs prior to prescribing the recombinant FVIII to be used for treatment. It is not essential to determine the actual mutations responsible for the hemophilia prior to prescribing the recombinant FVIII.

Two new haplotypes, H7 and H8, have been identified.

Based on the information that has been obtained, most white and Asian hemophiliacs should be treated with H1; black hemophiliacs should be tested for haplotype 1, 2 or 3, prior to treatment.

A transgenic animal model has been developed to testfornew diagnostic and therapeutics relating to hemophilia caused by intron 22 inversions.

### BRIEF DESCRIPTION OF THE DRAWINGS

FIGS. 1A, 1B and 1C are schematics of four nonsynonymous single-nucleotide polymorphisms (SNPs) whose haplotypes encode six distinct Factor VIII proteins, designated H1 through H6. Human F VIII contains four common nonsynonymous SNPs whose allelic combinations encode six distinct wild-type factor VIII proteins, only two of which have the amino acid sequences found in the recombinant factor VIII molecules used clinically. FIG. 1A shows a schematic illustration of both F VIII, with its 26 exons and 25 introns indicated by triangles and intervening lines, respectively, and factor VIII, with highlighting of its three A domains (A1, A2, and A3), single B domain (B), two C domains (C1 and C2), three acidic connecting peptides (a1, a2, and ap), and two immunodominant-inhibitor epitopes located in the A2 domain (oval) and the C2 domain (oval). By sequencing all 26 exons of the F VIII genes in 137 unrelated healthy persons from seven groups of diverse geographic origins, four nonsynonymous SNPs were identified: one in exon 10 (G1679A), two in exon 14 (A2554G and C3951G), and one in exon 25 (A6940G). These polymorphisms encode the following amino acid substitutions, respectively: histidine for arginine at position 484 (R484H), glycine for arginine at position 776 (R7760), glutamic acid for aspartic acid at position 1241 (D1241E), and valine for methionine at position 2238 (M2238V). The numbering systems used to designate the four nonsynonymous SNPs and the amino acid substitutions they encode are based on their nucleotide and residue locations, respectively, in the full-length F VIII complementary DNA and the mature circulating form of factor VIII. Whereas R776G and D1241E are located in the B domain, R484H and M2238V are components of the A2 and C2 immunodominant epitopes, respectively, which have been mapped to residues located at epitopes R484 to 1508 (isoleucine at position 508) and E2181 to V2243. FIG. 18 shows the six structurally distinct wild-type factor VIII proteins encoded by the naturally occurring allelic combinations (haplotypes) of the F VIII nonsynonymous SNPs G1679A, A25540, C3951G, and A69400. The amino acid residue at positions 484 (R or H), 776 (R or G), 1241 (D or E), and 2238 (M or V) are shown. The haplotype frequencies (f) listed for the six factor VIII proteins (H1 through H6) are based on their occurrence in 86 white (fwhite), 67 black (jblack), and 10 Chinese (fChinese) subjects. In FIG. 1C, the two full-length recombinant factor VIII proteins used in replacement therapy, Kogenate® and Recombinate®, contain the same amino acid sequences found in H1 (R-R-D-M) and H2 (R-R-E-M), respectively.

FIGS. 2A and 2B are schematics of hemophilic mutations and the four wild-type F VIII proteins predicted to be encoded by the background F VIII haplotypes on which they were identified. For factor VIII, the two immunodominant-inhibitor epitopes located in the A2 domain (oval) and the C2 domain (oval) are shown. Mutations found in patients with either an H1 or an H2 haplotype (H1+H2) are shown in FIG. 2A, and mutations found in patients with either an H3 or an H4 haplotype (H3+H4) are shown in FIG. 2B. For all haplotypes, missense mutations are shown above the appropriate factor VIII protein, and the other mutation types are shown below. Missense and nonsense mutations are indicated by their residue positions in the mature factor VIII protein. The point mutation T38039C, which occurs at position +2 of the 5′ splice site (SS) of intron 6, is designated according to the genomic nucleotide numbering system used for the F VIII reference sequence.17 The positions of four frameshift (FS)—inducing small deletions and insertions are numbered according to their locations in the full-length F VIII complementary DNA (c) with respect to the transcription start site. Specifically, one deletion (c.4292ATAGA) and three insertions (c.3809InsA, c.4551InsA, and c.4291InsATAGA) are indicated by the number of the wild-type nucleotide positioned immediately 5′ of the mutation site. AEx13 indicates an in-frame deletion of the 210-bp exon 13 sequence and an unknown amount of flanking nonexonic sequences from introns 12 and 13. For mutations that occurred in more than one patient, whether or not the patients were related, the number of times any given abnormality was observed (N) is indicated in parentheses. All previously unknown mutations are indicated with an asterisk. The baseline severity of hemophilia for each patient is shown by the color of the text defining his mutation, with black, blue, and red indicating severe, moderate, and mild disease, respectively. For mutations found in at least one inhibitor-positive (Inh[+]) patient, the nurnber of patients with a given abnormality in whom inhibitors developed is also indicated in parentheses. A 3′-terminal partial gene deletion involving exons 24, 25, and 26 in two inhibitor positive brothers is not shown.

### DETAILED DESCRIPTION OF THE INVENTION

### I. Definitions

As used herein, coagulation factor VIII (“F VIII”), is a coagulation factor present in normal plasma but deficient in the blood of persons with hemophilia A. It is a macromolecular complex composed of two separate entities, one of which, when deficient, results in hemophilia A, and the other, when deficient, results in von Willebrand's disease.

As used herein, hemophilia is a genetic disorder of blood clotting, caused by defective, inactive or missing F VIII, or by the presence of inhibitors to F VIII. Depending on the degree of the disorder present in an individual, excess bleeding may occur only after specific, predictable events (such as surgery, dental procedures, or injury), or occur spontaneously, with no known initiating event.

The normal mechanism for blood clotting is a complex series of events involving the interaction of the injured blood vessel, blood cells (called platelets), and over 20 different proteins which also circulate in the blood.

When a blood vessel is injured in a waythat Causes bleeding, platelets collect over the injured area, and form a temporary plug. to prevent further bleeding. This temporary plug, however, is too disorganized to serve as a long-term solution, so a series of chemical events occur, resulting in the foiuiation of a more reliable plug. The final plug involves tightly woven fibers of a material called fibrin. The production of fibrin requires the interaction of several chemicals, in particular a series of proteins called clotting factors. At least thirteen different clotting factors have been identified. The clotting cascade, as it is usually called, is the series of events required to form the final fibrin clot. The cascade uses a technique called amplification to rapidly produce the propersized fibrin clot from the small number of molecules initially activated by the injury.

In hemophilia, certain clotting factors are either decreased in quantity, absent, or improperly formed. Because the clotting cascade uses amplification to rapidly plug up a bleeding area, absence or inactivity ofjust one clotting factor can greatly increase bleeding time. Hemophilia A is the most common type of bleeding disorder and involves decreased activity of factor VIII. There are three levels of factor VIII deficiency: severe, moderate, and mild. This classification is based on the percentage of normal factor VIII activity present.

Individuals with less than 1% of normal factor VIII activity level have severe hemophilia. Half of all peoplewith hemophilia A fall into this category. Such individuals frequently experience spontaneous bleeding, most frequently into their joints, skin, and muscles. Surgery or trauma can result in life-threatening hemorrhage, and must be carefully managed. Individuals with 1-5% of normal factor VIII activity level have moderate hemophilia, and are at risk for heavy bleeding after seemingly minor traumatic injury. Individuals with 5-40% of normal factor VIII activity level have mild hemophilia, and must prepare carefully for any surgery or dental procedures.

Hemophilia A affects between one in 5,000 to one in 10,000 males in most populations. One study estimated the prevalence of hemophilia was 13.4 cases per 100,000 U.S. males (10.5 hemophilia A and 2.9-hemophilia B). By race/ethnicity, the prevalence was.13.2 cases/100,000 among white, 11.0 among African-American, and 11.5 among Hispanic males.

As used herein, a patient is considered to have an inhibitor if any screening assay ever had a value of 0.6 Bethesda units per milliliter or higher.

### I. Methods of Treatment

Infusion of plasma-derived onreconibinant factor VIII is the standard method of arresting hemorrhage in patients with hemophilia A (factor VIII deficiency). Alloantibodies that neutralize the activity of the replacement molecules develop in approximately 20 to 25% of patients, however, and the treatment of patients who have these inhibitors can be costly. The risk of formation of an inhibitor is influenced by the type of mutation in the factor VIII gene (F VIII). Large deletions, inversions, and nonsense mutations are associated with the highest risk, probably because the recipient's immune system recognizes the normal factor VIII replacement protein as a foreign molecule. The type of mutation also is associated with the severity of hemophilia A. Thus, the association between the type of mutation and the development of inhibitors may be confounded by variables related to the severity of illness, such as age at the first infusion of therapy or the cumulative number of days of replacement therapy.

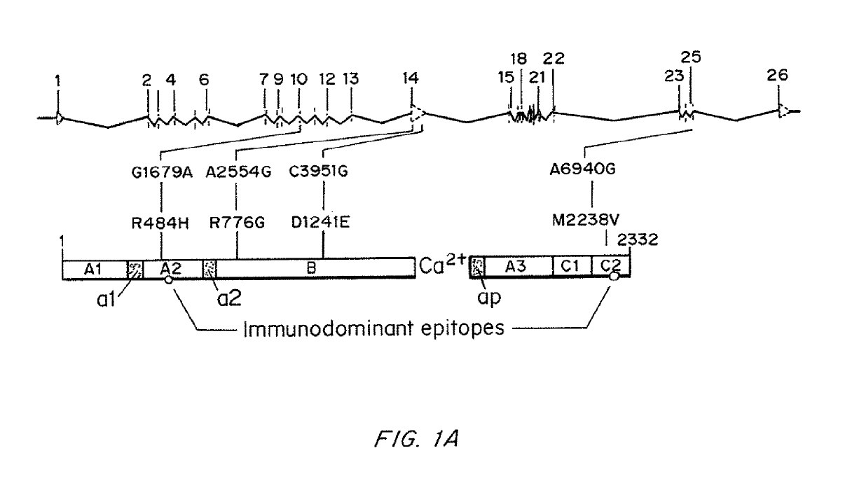

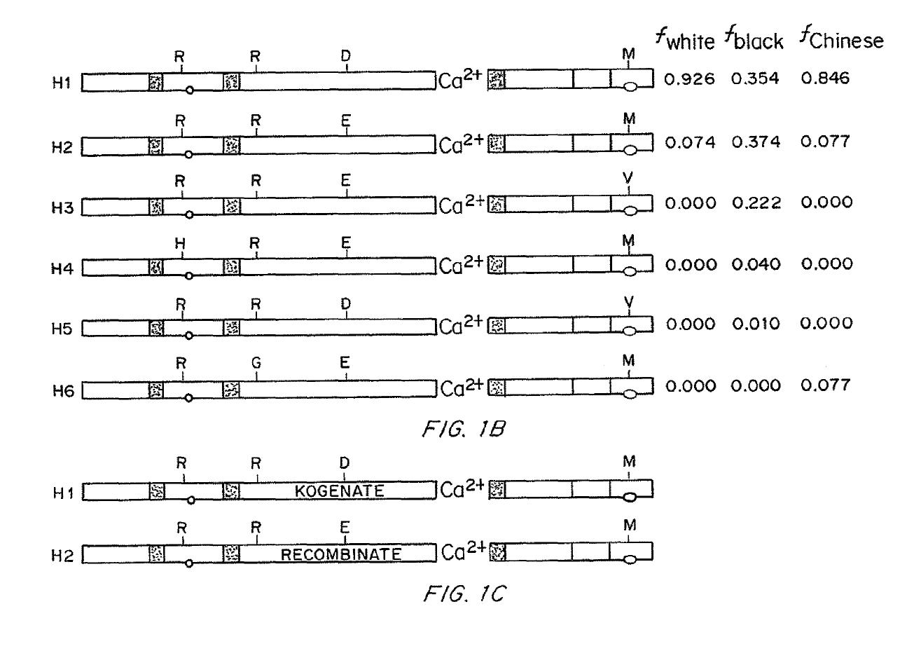

The prevalence of factor VIII inhibitors in black patients is about twice that in white patients. The mechanisms that account for this difference are unknown. In a study of F VIII in 137 healthy, unrelated people from seven groups of diverse geographic origins, four nonsynonyous single-nucleotide polymorphisms (SNPs)—61679A (encoding the amino. acid substitution of histidine for arginine at position 484 [R4841-1]), A2554G (encoding the substitution of glycine for arginine [R776G]), C3951G (encoding the substitution of glutamic acid for aspartic acid [D1241E]), and A6940G (encoding the substitution of valine for methionine [M2238V])⁻ whose haplotypes (allelic combinations) encode six distinct factor VIII proteins, which were designated H 1 through H6. Two of these proteins (H1 and H2) were found in all seven groups, but three (H3, H4, and H5) were found only in black people (16 subjects) and one (H6) was found only in Chinese people (10 subjects). (See FIG. 1.) The prevalence rates of H1 and H2 were 0.93 and 0.07, respectively, among whites in this study (86 subjects) and 0.35 and 0.37 among blacks. The prevalence rates of H3, H4, and H5 were 0.22, 0.04, and 0.01, respectively, among blacks. Kogenate (Bayer) and Recombinate (Baxter), the two full-length recombinant factor VIII products currently approved for use in persons with hemophilia A, correspond to the amino acid sequences of H 1 and H2, respectively.

In principle, therefore, one in four blacks with hemophilia A who require replacement therapy with recombinant factor VIII will receive products that differ from their own factor VIII protein at one or two residues, in addition to having amino acid differences attributable to the specific F VIII mutation. Plasma-derived factor VIII is also a source of exposure to H1 and H2, because most blood donors are white.

Therefore, in the preferred embodiment, black patients are haplotyped for haplotypes one, two or three, more preferably one, two, three, four or five, and then matched with the appropriate recombinant F VIII for treatment. The recombinant F VIII is administered in the same dosage and route of administration as is currently used with other commercially available recombinant F VIII formulations, such as Reconibinate® or Kogenate®.

Patients are sequenced accordingly to standard techniques, such as those described in the examples.

### II. Compositions for Treatment and Diagnosis

A. Compositions for Treatment

The compositions for treatment are recombinant F VIII, haplotype 1, 2, 3, 4, 5, 6, 7, or 8.

As shown in FIG. 1, the H1 and H2 haplotypes are represented by commercially available F VIII. While F VIII has previously been thought to be a monomorphic protein in the non-hemophilic population, there are at least six common non-synonymous-single-nucleotide polymorphisms (nsSNPs), combinations of which represent eight naturally-occurring allelic variants of the F VIII protein in the human population (FIG. 1). Combinations of four SNPs (R484H, R776G, D1241E, and M2238V) correspond to six of the eight haplotypes. This has been determined by direct DNA sequencing of PCR amplified fragments of the FVIII genes from numerous unrelated-individuals of multiple ethniCities. By examining male members of different ethnic groups (e.g. because they only have one X-Chromosome) and females who are homozygous for all nsSNPs or are only singly heterozygous, the naturally-occurring haplotypes (H) of these variations have been defined (e.g. the combinations by which the alleles of these five nsSNPs segregate naturally). As such, eight different haplotypic forms of the wt FVIII protein have been identified. The haplotypic forms that results from amino acid differences at R484H, R776G, D1241E, and M2238V have been designated: H1 (SEQ ID NO: 1) H2 (SEQ ID NO: 2), H3 (SEQ ID NO: 3), H4 (SEQ ID NO: 4), H5 (SEQ ID NO: 5), and H6 (SEQ ID NO: 6).

Two additional SNP's were,also identified as Q334P and R1260K. A haplotype designated H7 (SEQ ID NO:7 for cDNA and SEQ ID:8 for amino acid) is equivalent to the H1 haplotype except for a Q3 34P substitution. Another haplotype designated H8 (SEQ ID NO:9 for cDNA and SEQ ID NO:10 for amino acid) is equivalent to the H4 haplotype except for a R1260K substitution.

Each of these variants represents a normal allelic variant of the FVIII protein since the individuals from whom the sequences were described have no bleeding disorders.

The compositions are generally provided in lyophilized form which is reconstituted before use, then injected.

B. Compositions for Diagnosis

Kits for determining the haplotype of a hemophiliac include nucleic acid reagents specific for haplotype 1, 2, 3, 4, 5, 6, 7, or 8. In a preferred embodiment, the kit is for polymerase chain reaction and includes nucleic acid primers, controls (i.e., normal F VIII, known haplotypes), and other reagents for use in the reaction. In a preferred embodiment for diagnosis of the haplotype of black individuals in need of treatment, the kit includes reagents specific for haplotypes 1, 2 and 3. The kit may also include reagents for determining one or more mutations that cause hemophilia A.

### III. Transgenic Animal Models of Human FVIII

Transgenic animal models that express human F VIII transgenes are disclosed which are useful to testing of diagnostic and therapeutic agents for hemophilia. In some embodiments, the disclosed transgenic animals express human F VIII transgenes-that encode for human FVIII proteins with a haplotype such as an H1, H2, H3, H4, H5, H6, H7 or H8 haplotype.

In another embodiment, the transgenic animal expresses human FVIII containing an intron-22 inversion. The intron-22 inversion-may be present in the background of any of the disclosed human FVIII haplotypes. Although the recurrent intron-22 inversion, which accounts for almost half of all unrelated families with severe hemophilia-A. (Antonarakis, et al., Blood, 86:2206-12 (1995)), has been grouped together with large F VIII deletions and nonsense mutations into a “high risk” category with respect to inhibitor risk, alloimmunization to FVIII occurs in only about one in five patients with this frequently-observed gene abnormality overall. Moreover, in a few studies no patients with intron-22 inversions have developed-inhibitors. In light of these findings, the intron-22 inversion may not be an inherently high risk mutation type despite causing a cross reactive material (CRM)-negative (CRM-N) circulating FVIII deficiency, where plasma FVIII activity (FVIII:C) and antigen (FVIII:Ag) levels are both undetectable, and a severe bleeding diathesis, analogous to that caused by large deletions and nonsense mutations.

An intron-22 inverted F VIII allele cannot be transcribed into a full-length mRNA since the promoter region and most of the gene has been inverted. Thus exons 1 through 22 are transcribed as a polyadenylated fusion transcript in which two or more unrelated 3′-exons have replaced exons 23 through 26. This transcript therefore does not encode a full-length functional FVIII protein. However, the intrachromosomal homologous recombination causing the inversion also reconstitutes the F8B gene, which encodes a polyadenylated transcript with exons 23-26 spliced in-frame to an unrelated 5′-exon that has a Kozak consensus translation initiation codon. Therefore, the entire F VIII coding sequence is now contained within two mRNAs. In the FVIII producing cells of a patient with the intron-22 inversion, including the thymic epithelial cells that play a critical role in the normal physiologic processes that confer immunologic self-tolerance, these two mRNAs are translated into two polypeptide chains, which together contain the entire primary amino acid sequence of the FVIII protein. Since the process of becoming immunologically tolerant to a “self” protein requires that it first be translated intracelltilarly, it is believed that patients with intron-22 inversions could be tolerized to the specific polymorphic form of the FVIII protein encoded by their discontinuous FVIII exonic sequences. Patients with intron-22 inversions can be tolerized to the full-length (or B-domain deleted) FVIII protein encoded by the background haplotype of their F VIII gene before the inversion occurred. For example, a patient whose intron-22 inversion arose in a background F VIII haplotype encoding the most common black-restricted FVIII protein (H3) may be completely tolerized to an H3 replacement protein, which is not commercially available at present, but not to the two FDA approved replacement proteins Kogenate® (H1) and Recombinate® (H2). Similarly, a patient whose intron-22 inversion arose in a F VIII haplotype that encoded the most common FVIII protein in whites (H1) may be completely tolerized to Kogenate® but not to Recombinate®.

In certain embodiments, the transgenic animal is selected from the order Rodentia. Preferably, the transgenic animal is a mouse, although rats are also of particular utility. In other embodiments, the transgenic animal can be another mammal such as a pig or dog. Transgenic animals can be heterozygous or homozygous for the inserted transgene, but are preferably homozygous.

A. Transgenic Strategies

Transgenic animals expressing human FVIII proteins can be generated using any of several suitable strategies. In preferred embodiments, the expression of the F VIII gene endogenous to the recipient animal is disrupted so that the human F VIII transgene replaces the expression of the endogenous F VIII gene. This results in a transgenic animal that lacks functional activity of the endogenous FVIII protein, but possesses the functional activity of the human FVIII protein produced by the human F VIII transgene. The transgene can express any human F VIII gene, including any of the H1-H8 haplotypes disclosed herein. The human F VIII transgene can additionally contain an intron-22 inversion.

Transgenic animals that lack endogenous FVIII protein and express human FVIII proteins have several uses, including for research of in vivo functions for FVIII and as models for therapeutic intervention in FVIII-associated diseases and conditions, including Hemophelia A. For example, transgenic animals expressing specific haplotypes of human FVIII can be used to test if FVIII antibodies (“inhibitors”) are formed in response to replacement FVIII formulations.

Disruption of the endogenous F VIII gene is generally referred to as a gene “knock out”. A knock-out of an endogenous F VIII gene means that the function of the endogenous F VIII gene has been substantially decreased such that expression is not detectable or only present at insignificant levels. A “knock-in” transgenic animal refers to an animal that has had a modified gene introduced into its genome, wherein the modified gene can be of exogenous or endogenous origin. As used herein, “knock-in” transgenic animals encompasses animals in which an endogenous F VIII locus is replaced by a human F VIII locus the genome of that animal.

Knock-out and knock-in animals also include conditional knock-outs and conditional knock-ins. As used herein, “conditional” in reference to “knock-outs” and “knock-ins” means alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

In some embodiments, FVIII transgenic animals are generated by a cross between 1) an animal that is a knock-out for their endogenous F VIII gene and 2) an animal that expresses human F VIII and F8B genes. The animals generated from this cross lack expression of their endogenous F VIII gene and express human F VIII and F8B genes. F VIII knock-out animals can be generated by site-specific homologous recombination of a nucleic acid construct at the endogenous F VIII locus using standard methodologies, as described below. The recombination event can produce an endogenous F VIII gene that contains, for example, a deletion of the initiating ATG start codon and/or one or more functional domains necessary for FVIII activity. For example, F VIII knock-out mice have been generated by insertion of a selection cassette in exons 16 and 17 by homologous recombination (Bi, et al., Nat. Genet. (1995)). Animals expressing human F VIII and F8B genes can be generated by random integration of a construct containing human F VIII and F8B genes into the genome of the recipient animal. The construct containing the human F VIII and F8B genes can include promoter elements that regulate the expression of the genes in the recipient animal. In one embodiment, the promoter elements are the endogenous human F VIII and F8B promoter elements. In some embodiments, the human transgene is contained on a bacterial artificial chromosome (BAC) or a yeast artificial chromosome (YAC).

In other embodiments, FVIII transgenic animals are generated by knock-in of human F VIII and F8B genes at the locus of endogenous F VIII gene expression in the recipient animal using site-specific homologous recombination. This disrupts the expression of the endogenous F VIII gene of the recipient animal and at the same time replaces it with a human F VIII transgene of choice. In these animals, expression of the human F VIII transgene is under the control of the endogenous F VIII gene promoter of the recipient animal. In some embodiments, the human F VIII and F8B genes can be inserted into a bicistronic construct that encodes an internal ribosome entry site (TRES) and allows for expression of both genes under the control of the endogenous F VIII gene promoter of the recipient animal. In another embodiment, the promoter elements for the human F8B transgene are provided so that the human F VIII transgene is under the control of the F VIII promoter of the recipient animal and the human F8B transgene is under the control of human promoter elements.

In another embodiment, FVIII transgenic animals are generated by knock-in of a human F VIII gene at the locus of endogenous F VIII gene expression in the recipient animal using site-specific homologous recombination and insertion of a human F8B gene at a locus that causes results in constitutive expression of the F8B gene. For example, in mice, the F8B gene can be inserted at the Rosa26 permissive locus which drives ubiquitous, low-level expression of inserted genes.

B. Human F VIII Constructs

In one embodiment, the human F VIII nucleic acid construct is a targeting vector including two regions flanking the F VIII transgene wherein the regions are sufficiently homologous with portions of the genome of animal to undergo homologous recombination with the portions. Thus, targeting vectors for homologous recombination will include at least a portion of the human F VIII gene, and will include regions of homology to the target locus. DNA vectors for random-integration need not include regions of homology to mediate recombination. Conveniently, markers for positive and negative selection are included. Methods for generating cells having targeted gene modifications through homologous recombination are known in the art.

It is preferred that regions are selected to be of sufficient length and homology with portions of the genome to permit the homologous recombination of the transgene into at least one allele of the endogenous gene resident in the chromosomes of the target or recipient cell (e.g., ES cells). Preferably, the regions comprise approximately 1 to 15 kb of DNA homologous to the intended site of insertion into the host genome (more than 15 kb or less than 1 kb of the endogenous gene sequences may be employed so long as the amount employed is sufficient to permit homologous recombination into the endogenous gene).

In some embodiments, the nucleic acid construct comprises a selectable marker gene. In a preferred embodiment, the nucleic acid construct is a targeting vector including a selectable marker gene flanked on either side by regions that are sufficiently homologous with portions of the genome of the animal to undergo homologous recombination with those portions. In one embodiment, the portions of the genome correspond to sequences flanking or within the endogenous FVIII gene of the recipient animal. In this instance, the targeting vector is adapted to disrupt the endogenous gene.

The nucleic acid construct may contain more than One selectable maker gene. The selectable marker is preferably a polynucleotide which encodes an enzymatic activity that confers resistance to an antibiotic or drug upon the cell in which the selectable marker is expressed. Selectable markers may be “positive”; positive selectable markers typically are dominant selectable markers, i.e. genes which encode an enzymatic activity which can be detected in any animal, preferably mammalian, cell or cell line (including ES cells). Examples of dominant selectable markers include the bacterial aminoglycoside 3′ phosphoransferase gene (also referred to as the neo gene) which confers resistance to the drug G418 in animal cells, the bacterial hygromycin G phosphotransferase (hyg) gene which confers resistance to the antibiotic hygromycin and the bacterial xanthine-guanine phosphoribosyl transferase gene (also referred to as the gpt gene) which confers the ability to grow in the presence of mycophenolic acid. Selectable markers may be “negative”; negative selectable markers encode an enzymatic activity whose expression is cytotoxic to the cell when grown in an appropriate selective medium. For example, the Herpes simplex virus tk (HSV-tk) gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

More than one selectable marker gene may be employed with a targeting vector. In this instance, the targeting vector preferably contains a positive selectable marker (e.g. the neo gene) within the transgene and a negative selectable marker (e.g. HSV-tk) towards one or more of said outer regions flanking the transgene. The presence of the positive selectable marker permits the selection of recipient cells containing an integrated copy of the targeting vector whether this integration occurred at the target site or at a random site. The presence of the negative selectable marker permits the identification of recipient cells containing the targeting vector at the targeted site (i.e. which has integrated by virtue of homologous recombination into the target site); cells which survive when grown in medium which selects against the expression of the negative selectable marker do not contain a copy of the negative selectable marker.

The targeting vectors may include a recombinase system, which allows for the expression of a recombinase that catalyses the genetic recombination of a transgene. The transgene is flanked by recombinase recognition sequences and is generally either excised or inverted in cells expressing recombinase activity. In one embodiment, either the Cre-loxP recombinase system of bacteriophage P1 or the FLP recombinase system of Saccharomyces cerevisiae can be used to generate in vivo site-specific genetic recombination systems. Cre recombinase catalyses the site-specific recombination of an intervening target sequence or transgene located between loxP sequences. lox P sequences are 34 base pair nucleotide repeat sequences to which the Cre recombinase binds and are required for Cre recombinase mediated genetic recombination. The orientation of loxP sequences determines whether the intervening transgene is excised or inverted when Cre recombinase is present; catalysing the excision of the transgene when the loxP sequences are oriented as direct repeats and catalyses inversion of the transgene when loxP sequences are oriented as inverted repeats.

The vectors used in creating the transgenic animal may also contain other elements useful for optimal functioning of vector prior to or following its insertion into the recipient cell. These elements are well known to those of ordinary skill in the art. Preferably, the transgene components of the vector are assembled within a plasmid vector such as, for example, pBluescript (Stratagene) and then isolated from the plasmid DNA, prior to transformation of the target cells.

Vectors used for transforming mammalian embryos are constructed using methods well known in the art including without limitation the standard techniques of restriction endonuclease digestion, ligation, plasmid and DNA and RNA purification, DNA sequencing and the like as described, for example, in Sambrook, Fritsch and Maniatis, Eds., Molecular. A Laboratory Manual. (Cold Spring Harbor Laboratory Press, Cold Spring Harbor, N.Y. (1989)). Suitable vectors include, but are not limited to plasmids, retroviruses and other animal viruses, bacterial artificial chromosome (BACs), and yeast artificial chromosome (YACs).

C. Generation of Transgenic Animals

The disclosed transgenic animals are preferably generated by introduction of the targeting vectors into embryonal stem (ES) cells using techniques well known in the art: ES cells can be obtained by culturing pre-implantation embryos in vitro, under appropriate conditions using standard methodologies: Transgenes can be efficiently introduced into the ES cells by DNA transfection using a variety of methods known to the art including electroporation, calcium phosphate co-precipitation, protoplast or spheroplast fusion, lipofection and DEAF-dextran-mediated transfection. Transgenes may also be introduced into ES cells by retrovirus-mediated transduction or by microinjection. Such transfected ES cells can thereafter colonise an embryo following their introduction into the blastocoel of a blastocyst-stage embryo acid contribute to the germ line of the resulting chimeric animal. Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

Alternative methods for the generation of transgenic mammals are known to those skilled in the art. For example, embryonal cells at various developmental stages can be used to introduce transgenes for the production of transgenic mammals. Different methods are used depending on the stage of development of the embryonal cell. The zygote, particularly at the pronucleal stage (i.e., prior to fusion of the male and female pronuclei), is a preferred target for micro-injection. In the mouse, the male pronucleus reaches the size of approximately 20 micrometers in diameter, which allows reproducible injection of 1-2 picoliters (pl) of DNA solution. The use of zygotes as a target for gene transfer has a major advantage in that in most cases the injected DNA will be incorporated into the host genome before the first cleavage. As a consequence, all cells of the transgenic animal will carry the incorporated transgene. This will in general also be reflected in the efficient transmission of the transgene to offspring of the founder since 50% of the germ cells will harbour the transgene. Micro-injection of zygotes is the preferred method for random incorporation of transgenes.

Retroviral infection can also be used to introduce transgenes. The developing embryo cart be cultured in vitro to the blastocyst stage. During this time, the blastomeres can be targets for retroviral infection. Efficient infection of the blastomeres is obtained by treatment to remove the zona pellucida. The viral vector system used to introduce the transgene is typically a replication defective retrovirus carrying the transgene. Retroviral infection is easily and efficiently obtained by culturing the blastomeres on a monolayer of virus-producing cells. Alternatively, infection can be performed at a later stage. Virus or virus-producing cells can be injected into the blastocoele. Most of the founders will be mosaic for the transgene since incorporation occurs only in a subset of cells which form the transgene mammal. Further, the founder may contain various retroviral insertions of the transgene at different positions in the genome, which generally will segregate in the offspring. In addition, it is also possible to introduce transgenes into the germline, albeit with low efficiency, by intrauterine retroviral infection of the midgestation embryo. An additional means of using retroviruses or retroviral vectors to create transgenic mammals known to the art involves the micro-injection of retroviral particles or mitomycin C-treated cells producing retrovirus into the perivitelline space of fertilised eggs or early embryos.

In selecting lines of any mammalian species, they may be selected for criteria such as embryo yield, pronuelear visibility in the embryos, reproductive fitness, color selection of transgenic offspring, or availability of ES cell clones. For example, if transgenic mice are to be produced, lines such as C57/B16 or 129 may be used.

The age of the mammals that are used to obtain embryos and to serve as surrogate hosts is a function of the species used. When mice are used, for example, pre-puberal females are preferred as they yield more embryos and respond better to hormone injections.

Administration of hormones or other chemical compounds may be necessary to prepare the female for egg production, mating and/or implantation of embryos. Usually, a primed female (i.e. one that is producing eggs that may fertilised) is mated with a stud male and the resulting fertilised embryos are removed for introduction of the transgene(s). Alternatively, eggs and sperm may be obtained from suitable females and males and used for in vitro fertilisation to produce an embryo suitable for introduction of the transgene.

Normally, fertilised embryos are incubated in suitable media until the pronuclei appear. At about this time, the exogenous nucleic acid sequence comprising the transgene of interest is introduced into the male or female pronucleus. In some species, such as mice, the male pronuelease is preferred.

Introduction of nucleic acid maybe accomplished by any means known, in the art such as, for example, microinjection. Following introduction of the nucleic acid into the embryo, the embryo maybe incubated in vitro for varied amounts of time prior to reimplantation into the surrogate host. One common method is to incubate the embryos in vitro for 1 to 7 days and then reimplant them into the surrogate host.

Reimplantation is accomplished using standard methods. Usually the surrogate host is anaesthetised and the embryos are inserted into the oviduct. The number of embryos implanted into a particular host will vary, and will usually be comparable to or higher than the number of offspring the species naturally produces. Transgenic offspring of the surrogate host may be screened for the presence of the transgene by any suitable method. Screening may be accomplished by Southern or northern analysis using a probe that is complementary to at least a portion of the transgene (and/or a region flanking the transgene) or by PCR using primers complementary to portions of the transgene (and/or a region flanking the transgene). Western blot analysis using an antibody against the protein encoded by the transgene may be employed as an alternative or additional method for screening.

Alternative or additional methods for evaluating the presence of the transgene include without limitation suitable biochemical assays such as enzyme and/or immunological assays, and histological stains for particular markers or enzyme activities.

Progeny of the transgenic mammals may be obtained by mating the transgenic mammal with a suitable partner or by in vitro fertilisation using eggs and/or sperm obtained from the transgenic mammal. Where in vitro fertilisation is used, the fertilised embryo is implanted into a surrogate host or incubated in vitro or both. Where mating is used to produce transgenic progeny, the transgenic mammal may be back-crossed to a parental line, otherwise inbred or cross-bred with mammals possessing other desirable genetic characteristics. The progeny maybe evaluated for the presence of the transgene using methods described above, or other appropriate methods.

The present invention will be further understood by reference to the following non-limiting examples.

### EXAMPLES

### Example 1

### Determination of Association of Haplotype and Ethnicity with Different Mutations Causing Hemophilia

Black patients with hemophilia A (factor VIII deficiency) are twice as likely as white patients to produce inhibitors against factor VIII proteins given as replacement therapy. There are six wild-type factor VIII, proteins, designated H1 through H6, but only two (H1 and H2) match the recombinant factor VIII products used clinically. H1 and H2 are found in all racial groups and are the only factor VIII proteins found in the white population to date. H3, H4, and H5 have been found only in blacks. It was hypothesized that mismatched factor VIII transfusions contribute to the high incidence of inhibitors among black patients.

Methods

The factor VIII gene (F VIII) in black patients with hemophilia A was sequenced to identify causative mutations and the background haplotypes on which they reside. Results from previous Bethesda assays and information on the baseline severity of hemophilia, age at enrollment, and biologic relationships among study patients were obtained from review of the patients' medical charts. Multivariable logistic regression was used to control for these potential confounders while testing for associations between F VIII haplotype and the development of inhibitors.

### Patients

Black patients with hemophilia A undergoing treatment at any of four Federal Region IV South Hemophilia Treatment Centers were invited to participate in this study during scheduled annual visits. The participating centers were Emory University; Atlanta; the University of Alabama at Birmingham, Birmingham; the Medical College of Georgia, Augusta; and the University of Mississippi Medical Center, Jackson. Each of the 78 enrolled patients provided a blood sample. Patients or their parents or legal guardians gave written informed consent for participation in the study. The institutional review boards of each participating center approved the protocol.

A short, standardized survey was administered to all patients by each center. Information concerning self-reported race, age, baseline severity of hemophilia, results of previous testing for inhibitors, and other affected family members was obtained from medical records and interviews with patients by the nurses involved with enrollment. To take into account nonindependence of subjects due to family relationships, all patients with affected relatives were asked whether any relative was being treated at any of the participating centers and thus might be enrolled in this study.

Inhibitor Surveillance and Determination of Baseline Severity of Hemophilia

Data on inhibitors were obtained from reviews of the medical charts by the nurses. To identify inhibitors, the participating centers used the Bethesda assay with a Nijmegen modification known to improve its specificity near the cutoff for a positive test result, which was 0.6 Bethesda unit per milliliter. In general, patients were screened for inhibitors during their annual evaluations. Baseline severity of hemophilia was defined according to the initial level (in units per milliliter) of factor VIII activity as a percentage of normal. Mild hemophilia corresponded to a baseline level of factor VIII greater than 5% but less than 40% of normal, moderate hemophilia to a baseline level equal to or greater than 1% but no greater than 5% of normal, and severe hemophilia to .a baseline level less than 1% of normal. To measure factor VIII, each center used factor VIII-deficient plasma and assessment of the activated partial-thromboplastin time.

### F VIII Sequencing

All known functional regions of F VIII, including 1194 bp of the contiguous promoter sequence, all 26 exons, 50 to 100 bp of each junctional-intronic segment, and 309 by of flanking 3′-genomic DNA, were amplified by the polymerase chain reaction (PCR) and sequenced. Sequencing was performed to genotype the known nonsynonymous SNPs, discover new nonsynonymous SNPs, and identify the noninversion hemophilia-causing mutations. The sequencing chromatogram were processed with Phred software (www.phrap.org) and SAS software programs and were then reviewed manually. Given that males have only one X chromosome, patients with hemophilia are hemizygous for F VIII, and thus haplotypes were constructed as a simple combination of the patient's nonsynonymous SNP alleles.

### F VIII Inversion Assays

Genomic DNA samples and slightly modified versions of three PCR-based assays to identify inversions in introns 1 and 22. Patients whose F VIII mutations were not identified definitively by sequencing were evaluated for the intron 22 inversion by long-range PCR. Unless an intron 22 inversion was definitively identified, the Outcome, Exposure, and Covariates intron 1 inversion assay was performed (Bagneil, et al., Blood 2002; 99:168-74). A patient was considered to have an inhibitor unless an intron 1 inversion was definitely identified or a screening assay ever had a value of 0.6 Bethesda or a more robust inverse-PCR-based intron 22 unit per milliliter or higher.

The background wild-type form of the factor VIII protein encoded by a patient's F VIII gene was determined on the basis of specified amino acid residues at positions 484 (R or H), 776 (R or G), 1241 (D or E), and 2238 (M or V). On the basis of the alleles of G1679A, A2554G, C3951G, and A6940G, the background F VIII haplotypes identified in this study were predicted to encode four of the five wild-type factor VIII proteins observed previously in the black population, namely, H1, H2, H3, and H4 (FIG. 1A). Because of the small number of subjects, they were combined into two groups: H1 with H2 (H1+H2) and H3 with H4 (H3+H4). Patients in the H1+H2 group represent nonexposed (control) subjects, since their hemophilic mutations are present in F VIII haplotypes that encode the factor VIII proteins represented by-or enriched in recombinant and plasma-derived replacement products. The H3+H4 group is composed of exposed (case) subjects, since their F VIII mutations reside within haplotypes encoding the black-restricted factor VIII proteins H3 and H4, which are structurally distinct from, and therefore mismatched with, the recombinant (and plasma-derived) factor VIII products used clinically. In an unadjusted analysis, the prevalence of inhibitors was significantly different among patients grouped according to their factor VIII haplotypes. Logistic-regression analysis with control for age at enrollment and baseline severity of illness was performed and these analyses repeated in the subgroup of subjects with hemophilia-causing missense mutations only. SAS software for Windows, version 9.1.3, was used for all statistical analyses.

Accounting for Nonindependence Due to Family Relationships

Because the study questionnaire identified several related patients, there was a concern that association of the development of inhibitors with F VIII haplotype might be due to the fact that family members, who share the same haplotype, are also more likely to share alleles of other polymorphic loci, including those that may influence the development of inhibitors, such as the genes for tumor necrosis factor α and interleukin-10. Therefore patients without affected relatives were enrolled in the study as singletons and grouped those with reported affected relatives into pedigrees. A series of both crude and adjusted sub-analyses were performed after progressing through all combinations of unrelated subjects, selecting only one member from each family that had more than one affected member, and recorded the resulting odds ratios.

### Results

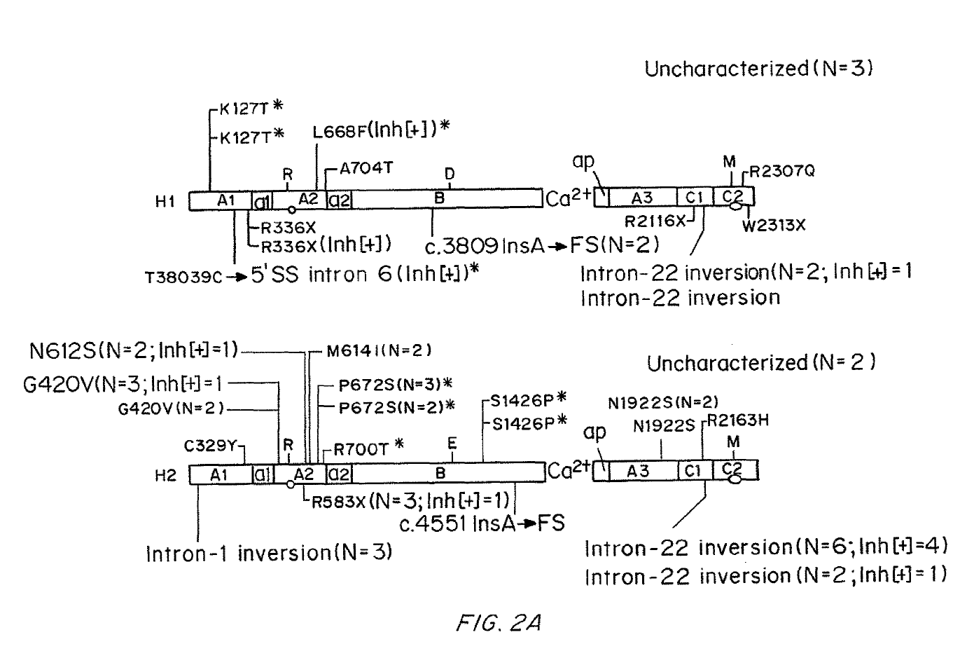

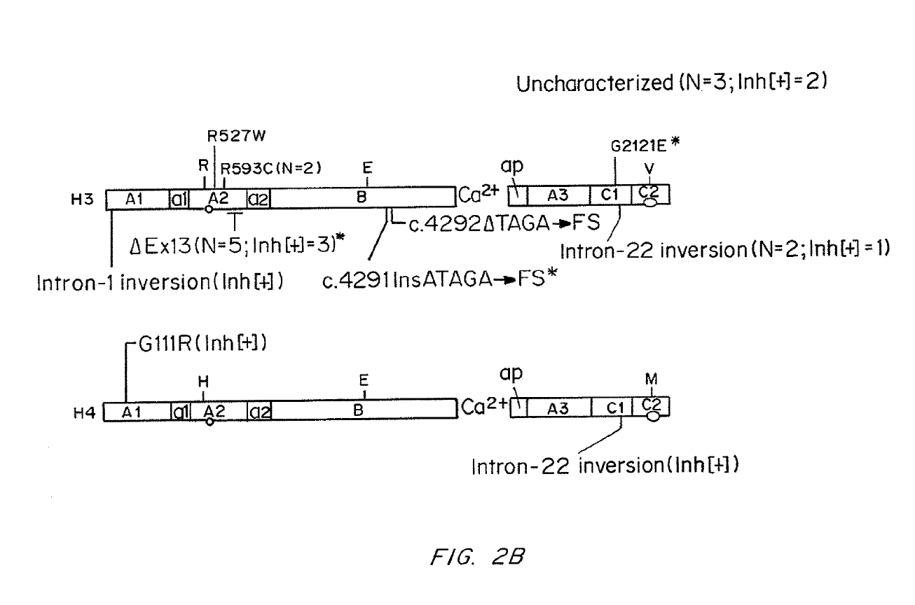

Seventy-eight black patients with hemophilia A were enrolled. The hemophilic F8mutation was identified in 70 of the 78 patients (FIG. 2A). Two full brothers with large gene deletions that-included A6940G, the nonsynonymous SNP encoding M223 8V (Table 1), were excluded from the association analyses, since they could not be classified within either haplotype group. The mean (±SD) age of the 76 remaining subjects was 17.5±12.9 years. According to initially recorded measurements of factor VIII, 11 subjects (14%) had mild hemophilia, 17 (22%) had moderate hemophilia, and 48 (63%) had severe hemophilia; this distribution of severity is similar to that seen in cross-sectional. studies of patients with hemophilia from other racial groups.

In the black patients with hemophilia, haplotypes H1, H2, H3, and H4 were identified, but not the infrequent H5 haplotype. Two patients had one additional, previously unknown nonsynonymous SNP, neither of which was predicted to cause hemophilia. The frequencies of mild, moderate, and severe hemophilia did not differ significantly according to the four background haplotypes (P=0.11). Table 2 shows the relationship between haplotype group and the prevalence of inhibitors. The odds of having a factor VIII inhibitor were significantly higher among patients with an H3 or H4 haplotype than among those with an H1 or H2 haplotype (odds ratio, 3.4; 95% confidence interval [CI], 1.1 to 10.2; P=0.03). This association remained when we controlled for age at enrollment and baseline severity of hemophilia in a multivariable logistic regression (odds ratio, 3:6; 95% CI, 1.1 to 12.3; P=0:04).

The two patients whose F VIII genes had different background haplotypes were excluded because of the presence of one additional nonsynonymous SNP each. Of the remaining 74 patients, 51 had no reported relative among the study participants. The other 23 patients were members of 11 families. When a single patient was selected from each of these families, the sample size for the subanalysis was 62 patients. In analyses of all 3072 possible combinations of 62 unrelated persons, the median odds ratios for the development of factor VIII inhibitors were 2.5 and 2.6 in the unadjusted and adjusted analyses, respectively. The maximum and minimum odds ratios observed in any single sub-sample of unrelated persons were 4.3 and 1.5, respectively, in the unadjusted analysis and 4.4 and 1.5 in the adjusted analysis.

Table 1 shows that 11 different categories of hemophilic mutation types were identified in the 78 black patients. These 11 mutation categories consisted of 31 distinct loss-of-function F VIII alleles, 9 of which were previously unknown (FIG. 2). This large degree of allelic heterogeneity is similar to what has been observed in previous cross-sectional studies to identify the mutational spectrums in patients from other racial groups. Among the 70 patients with identified F VIII mutations, no difference was observed between the H1+H2 and the H3+H4 haplotype comparison groups in the proportion of patients with higher-risk or lower-risk types of mutation (P=0.27) (Table 1). To reduce the heterogeneity of the unknown effects of different mutation types, a subanalysis was conducted among patients with missense mutations (the only category large enough to yield a meaningful result), using multivariable logistic -regression to control for age at enrollment and baseline severity of illness. The prevalence of inhibitor development was higher in those whose missense mutations resided on a haplotype encoding H3 or H4 (odds ratio, 4.3), although the confidence interval in this small sub-sample of 31 patients included the possibility of a null effect (95% CI, 0.2 to 101.1).

Summary an&Conclusion

Of the 78 black patients with hemophilia enrolled, 24% had an H3 or H4 background haplotype. The prevalence of inhibitors was higher among patients with either of these haplotypes than among patients with haplotype H1 or H2 (odds ratio, 3.6; 95% confidence interval, 1.1 to 12.3; P=0.04), despite a similar spectrum of hemophilic mutations and degree of severity of illness in these two subgroups. These indicate that mismatched factor VIII replacement therapy is a risk factor for the development of anti-factor VIII alloantibodies.

Previous investigations of nonhemophilic populations (FIG. 1) led to the prediction that the causative hemophilic mutations in approximately 27% of black patients would be present on background F VIII haplotypes encoding either the H3, H4, or H5 wild-type forms of factor VIII. These haplotypes differ from the H1 and H2 proteins. H1 and H2 proteins constitute the currently available recombinant factor VIII products and are enriched in plasma-derived factor VIII concentrates, since blood donors in the United States are predominantly white. It was found that patients with either an H1 or an H4 background haplotype were more likely to have an inhibitor (at some point in their lives) than were patients whose haplotypes were either H1 or H2. Only black patients were studied in this study to reduce the magnitude of any potential confounding variables due to population stratification across the haplotype comparison groups. Because the sample size was small, patients were combined into two groups, H1+H2 and H3+H4. The amino acid sequences of the background H1 and H2 proteins correspond to the full-length recombinant factor VIII molecules (FIG. 1C) and the two factor VIII proteins predicted to predominate in existing plasma-derived products. There was insufficient information about the brands of concentrate to the patients had been exposed to compare the prevalence of inhibitor development between patients with an H1 haplotype who had been treated with Kogenate® (the H1 molecule) and those treated with Recombinate® (the H2 molecule), or, conversely, between patients with an H2 haplotype who had been treated with one or the other concentrate. Thus, in this study, the inclusion of all H1 and H2 patients in the reference group could result in a bias toward the null (i.e., it could bring the odds ratio closer to 1 than its true value). Of the three non-synonymous SNPs whose encoded amino acid residues distinguish H3 and H4 from H1 and H2, two are located in immunodominant epitopes (R484 to I508 [isoleucine at position 508] and E2181 to V2243), sites at which neutralizing factor VIII alloantibodies from most patients with inhibitors interact. In the multivariable regression analysis, there was insufficient data to control for some potentially important variables, such as previous exposure to plasma-derived or recombinant factor VIII products (or both), cumulative days of exposure, age at first infusion, or whether the inhibitors that developed were transient or permanent and of low or high titer. The distribution of allelic variants of immune-response genes associated with the development of inhibitors in the two haplotype groups were not compare. Age at enrollment and baseline severity of hemophilia were controlled for, but these are at best poor surrogates for age at first infusion and cumulative days of exposure. A conservative approach was used to account for the effect of related patients, by selecting only one member from each family and progressing through all possible combinations of unrelated persons. The average odds ratios in these analyses differed little from those found for the overall sample. Thus, the presence of some related patients in the study was probably not a source of bias.

TABLE 1
Factor VIII Gene Mutation Type According to Background
Factor VIII Protein Haplotype in 78 Black Patients with Hemophilia A.*

| | Reported | | | |
|---|---|---|---|---|
| | Inhibitor | | | H3 + |
| | Prevalence‡ | Total§ | H1 + H2§ | H4§ |
| Mutation Type† | % | no. (%) | | |
| Higher risk for inhibitors | Not known | 57 (73) | 40 (70)¶ | 15 (79)¶ |
| Large deletions of ≧2 | 68.2-88.0 | 2 (3) | 0 | 0 |
| exons (>1 domain)* | | | | |
| Nonsense mutations (light | 40.0-50.0 | 2 (3) | 2 (4) | 0 |
| chain) | | | | |
| Intragenic inversions | 21.0-35.0 | 14 (18) | 11 (19) | 3 (16) |
| (intron 22) | | | | |
| Large deletions of ≦1 | 11.9-25.0 | 5 (6) | 0 | 5 (26) |
| exon (≦1 domain)□ | | | | |
| Small insertions or | 20.6-21.0 | 2 (3) | 0 | 2 (11) |
| deletions (non-A-run) | | | | |
| Nonsense mutations (heavy | 14.3-17.0 | 5 (6) | 5 (9) | 0 |
| chain) | | | | |
| Intragenic inversions | 10.0-17.0 | 4 (5) | 3 (5) | 1 (5) |
| (intron 1) | | | | |
| Missense mutations (A2, | 10.0-12.0 | 23 (29) | 19 (33) | 4 (21) |
| C1, and C2 domains) | | | | |
| Lower risk for inhibitors | Not known | 13 (17) | 12 (21) | 1 (5) |
| Splice-site mutations | 2.2-17.0 | 1 (1) | 1 (2) | 0 |
| Small insertions or | 3.0-6.0 | 3 (4) | 3 (5) | 0 |
| deletions (A-run) | | | | |
| Missense mutations (other | 3.0-3.9 | 9 (12) | 8 (14) | 1 (5) |
| regions) | | | | |
| Not identified | Not | 8 (10) | 5 (9) | 3 (16) |
| | applicable | | | |
| Total | Not | 78 | 57 | 19 |
| | applicable | | | |

*Two inhibitor-positive brothers with a deletion of exons 24, 25, and 26 (a large deletion) could not be assigned to either the H1 + H2 or the H3 + H4 group, since A6940G, which encodes M2238V, is located in exon 25.
†11 different mutation types identified.
‡The range of reported prevalence rates of inhibitors for each mutation type is given.^{3,37,41}
§The number and percentage of patients with any given mutation type in the overall study cohort and either the nonexposed (H1 + H2) or the exposed (H3 + H4) group is given.
¶The proportion of patients with higher-risk mutation types does not differ significantly between the H1 + H2 and the H3 + H4 haplotype groups (P = 0.27 by two-sided Fisher's exact test). An in-frame deletion of exon 13 (ΔEx13) is predicted in five patients on the basis of a repeated failure of multiple independent polymerase chain reactions to generate the appropriate amplicon only when genomic DNAs from these patients were used.

TABLE 2
Development of Inhibitors to Factor VIII According to Factor
VIII Haplotype among 76 Black Patients with Hemophilia A.

| | Development of | | |
|---|---|---|---|
| | Factor VIII Inhibitor | | |
| | Yes | No | Odds Ratio |
| Variable | no. of patients | | (95% CI) |
| Factor VIII haplotype | | | |
| H4 | 2 | 0 | Undefined* |
| H3 | 7 | 10 | 2.5 (0.6-10.7)† |
| H2 | 8 | 31 | 0.9 (0.2-3.5)† |
| H1 | 4 | 14 | Reference group |
| Haplotype group | | | |
| H3 + H4 | 9 | 10 | 3.4 (1.1-10.2)‡ |
| H1 + H2 | 12 | 45 | |

*P = 0.79 by two-sided Fisher's exact test.
†The odds of having a factor VIII inhibitor were not significantly higher among the H3 or H2 patients alone than among the H1 patients.
‡The odds of having a factor VIII inhibitor were significantly higher among patients in the H3 + H4 group than among those in the H1 + H2 group.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treatment of a hemophiliac consisting of:
(a) determining the haplotype of the hemophiliac, and
(b) prescribing or administering factor VIII of the determined haplotype to the hemophiliac.

2. The method of claim 1 further comprising determining the one or more mutations that cause the hemophilia.

3. The method of claim 1 wherein the haplotype is selected from the group consisting of haplotype 1, 2, 3, 4, 5, 6, 7 and 8.

4. The method of claim 1 wherein the hemophiliac is a black patient, comprising determining if the hemophiliac has haplotype 1, 2, 3, 4, 5, 6, 7 or 8;

5. The method of claim 4 comprising determining if the hemophiliac has haplotype 1, 2, or 3, then prescribing a factor VIII of the same haplotype.

6. A purified or isolated haplotype 7 Factor VIII.

7. A purified or isolated haplotype 8 Factor VIII.

8. A kit for determining the haplotype of a hemophiliac comprising nucleic acid reagents specific for haplotypes selected from the group consisting of haplotype 1, 2, 3, 4, 5, 6, 7, and 8.

9. The kit of claim 8 for polymerase chain reaction comprising nucleic acid primers.

10. The kit of claim 8 comprising reagents specific for haplotypes 1, 2 and 3.

11. The kit of claim 8 further comprising reagents for determining one or more mutations that cause hemophilia A.

12. A transgenic animal expressing-human FVIII.

13. The animal of claim 12 wherein the FVIII contains an intron-22 inversion.

14. The animal of claim 12 wherein the human FVIII has a haplotype selected from the group consisting of haplotype 1, 2, 3, 4, 5, 6, 7 and 8.

15. The animal of claim 13 wherein the human FVIII has a haplotype selected from the group consisting of haplotype 1, 2, 3, 4, 5, 6, 7 and 8.
